(19) United States
(12) Patent Application Publication
(10) Pub. No.: US 2025/0308685 A1
(43) Pub. Date: Oct. 2, 2025

(54) VIDEO ANALYTICS FOR MEDICAL DEVICE IMPROVEMENTS

(71) Applicant: Welch Allyn, Inc., Skaneateles Falls, NY (US)

(72) Inventors: Gene J. Wolfe, Pittsford, NY (US); John A. Lane, Venice, FL (US); Craig M. Meyerson, Syracuse, NY (US); WonKyung McSweeney, Manlius, NY (US); David E. Quinn, Moravia, NY (US)

(21) Appl. No.: 19/086,588

(22) Filed: Mar. 21, 2025

Related U.S. Application Data
(60) Provisional application No. 63/570,398, filed on Mar. 27, 2024.

(51) Int. Cl. G16H 40/40; G16H 30/40

(52) U.S. Cl. CPC G16H 40/40; G16H 30/40

(57) ABSTRACT

A system for improving medical device performance is disclosed. The system receives video data of a patient environment where at least one medical device is positioned. The system analyzes the video data to determine a status of the at least one medical device. The system adjusts an operation of the at least one medical device based on the status. The system can further monitor the video data over a period of time to determine classifications of a plurality of caregivers who enter and exit the patient environment over the period of time, generate metrics based on the classifications of the plurality of caregivers, and adjust the operation of at least one medical device based on the metrics.

### CROSS-REFERENCE TO RELATED APPLICATION

This application claims the benefit of and priority to U.S. Provisional Patent Application No. 63/570,398, filed Mar. 27, 2024, the entire disclosure of which is incorporated by reference herein in its entirety.

### BACKGROUND

Healthcare organizations often desire the ability to monitor, allocate, and forecast consumption of resources such as usage of medical devices and other resources in order to make informed decisions regarding facility operational planning. However, it is difficult to track the consumption of resources in busy environments such as hospitals. Additionally, it can be even more difficult to bring about operational changes to utilize resources more efficiently.

### SUMMARY

In general terms, the present disclosure relates to video analytics for medical device improvements. In one possible configuration, operation of at least one medical device is adjusted based on a status determined from video data of a patient environment. Various aspects are described in this disclosure, which include, but are not limited to, the following aspects.

One aspect relates to a system for improving medical device performance, the system comprising at least one processing device; and at least one computer readable data storage device storing software instructions that, when executed by the at least one processing device, cause the at least one processing device to: receive video data of a patient environment where at least one medical device is positioned; analyze the video data to determine a status of the at least one medical device; and adjust an operation of the at least one medical device based on the status.

Another aspect relates to a method of improving medical device performance, the method comprising: receiving video data of a patient environment where at least one medical device is positioned; analyzing the video data to determine a status of the at least one medical device; and adjusting an operation of the at least one medical device based on the status.

Another aspect relates to a system for improving medical device performance, the system comprising: a camera for recording video data of a patient environment; at least one processing device; and at least one computer readable data storage device storing software instructions that, when executed by the at least one processing device, cause the at least one processing device to: monitor the video data over a period of time to determine classifications of a plurality of caregivers who enter and exit the patient environment over the period of time; generate metrics based on the classifications of the plurality of caregivers; and adjust an operation of at least one medical device based on the metrics.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a healthcare facility that includes a video analytics system for monitoring medical devices, caregivers, and other resources.

FIG. 2 schematically illustrates an example of the video analytics system of FIG. 1.

FIG. 3 schematically illustrates an example of a clinical analytics module and an operational analytics module implemented on the video analytics system of FIG. 2.

FIG. 4 schematically illustrates an example of a method of improving medical device performance in the healthcare facility of FIG. 1.

FIG. 5 schematically illustrates an example of a central analytics repository that can be managed by the video analytics system of FIG. 1.

FIG. 6 schematically illustrates an example of a method of benchmarking metrics determined for the healthcare facility of FIG. 1.

FIG. 7 illustrates examples of caregivers that can be classified by the video analytics system of FIG. 1.

FIG. 8 schematically illustrates an example of a method of improving healthcare provided in the healthcare facility of FIG. 1.

FIG. 9 schematically illustrates an example of a method of improving usage of the medical devices in the healthcare facility of FIG. 1.

### DETAILED DESCRIPTION

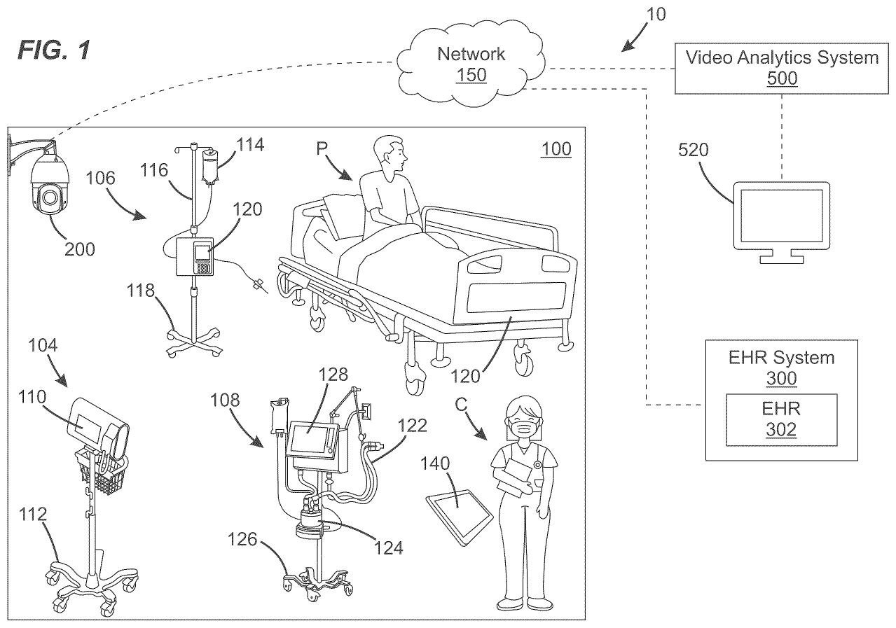

FIG. 1 illustrates an example of a healthcare facility 10 that includes a video analytics system 500 for monitoring medical devices, caregivers, and other resources. In at least some examples, the video analytics system 500 is part of a virtual nursing system for monitoring patients inside a plurality of patient environments in the healthcare facility 10. The video analytics system 500 augments the virtual nursing system with additional analysis modules that employ algorithms that measure in real-time metrics related to the healthcare provided to the patients in the healthcare facility 10. Examples of the healthcare facility 10 can include hospitals, long-term care facilities, nursing homes, surgery centers, and the like.

As will be described in more detail, the video analytics system 500 determines statuses of medical devices and caregivers in the healthcare facility 10. The determined statuses can be used to allocate resources more efficiently within the healthcare facility 10. Further, the determined statuses can be used to adjust operations of the medical devices such that the medical devices are used more efficiently in the healthcare facility 10. Additionally, the video analytics system 500 determines classifications of caregivers inside patient environments.

In FIG. 1, a patient P is shown resting on a patient support apparatus 102 inside a patient environment 100 of the healthcare facility 10. As an illustrative example, the patient environment 100 is a patient room in a hospital. As a further example, the patient room is within a med-surg unit or floor of the hospital. As another example, the patient environment 100 is an operating room in the hospital. The healthcare facility 10 includes a plurality of patient environments 100 such that the patient environment 100 is provided for illustrative purposes.

The patient environment 100 includes medical devices and equipment such as the patient support apparatus 102, and can include additional types of medical devices such as a patient monitoring device 104, an infusion pump 106, and a ventilator 108. Additional types of medical devices, or fewer types of medical devices can be positioned inside the patient environment 100, such that the patient support apparatus 102, the patient monitoring device 104, the infusion pump 106, and the ventilator 108 are shown for illustrative purposes.

The patient support apparatus 102 can be a hospital bed, a stretcher, an operating room table, or similar type of apparatus on which the patient P can rest. The patient support apparatus 102 can include one or more sensors that measure one or more physiological parameters of the patient P such as heart rate, non-invasive blood pressure (NIBP), motion, and weight. Additionally, the patient support apparatus 102 can include sensors that detect patient exit, incontinence, deterioration, and other metrics relevant to the health of the patient P.

The patient monitoring device 104 can be used to measure and monitor physiological parameters of the patient P, and to display representations of the measured physiological parameters on a display 110. In some examples, the display 110 is a touchscreen that operates to receive tactile inputs from a user such as the caregiver C such that the display 110 is both a display device and a user input device. In some examples, the display 110 is a liquid-crystal display (LCD), an organic light-emitting diode (OLED, a plasma panel, a quantum-dot light-emitting diode (QLED), or other type or combination of display screen technology.

The patient monitoring device 104 includes one or more sensor modules that can be used to measure one or more physiological parameters of the patient P. For example, the patient monitoring device 104 can include a temperature sensor module for measuring the patient P's temperature, a pulse oximetry sensor module for measuring the patient P's blood oxygen saturation (SpO2), and a non-invasive blood pressure (NIBP) sensor measurement module for measuring the patient P's blood pressure. As used herein, a “module” is a combination of physical structure which resides in the patient monitoring device 104 and peripheral components that attach to and reside outside of the patient monitoring device 104. The patient monitoring device 104 can include additional sensor modules for receiving additional physiological parameter measurements, including heart rate, pulse, and ECG/EKG.

In the illustrative example shown in FIG. 1, the patient monitoring device 104 is mounted on a mobile cart 112 such that the patient monitoring device 104 is portable and can be brought into and out of the patient environment 100. In alternative examples, the patient monitoring device 104 can be stationary such that it can include a wall mounted unit.

The infusion pump 106 controls intravenous delivery of a fluid from a container 114 to the patient P. The infusion pump 106 is shown mounted on a mobile cart 118 such that the infusion pump 106 is portable and can be brought into and out of the patient environment 100 as needed. The mobile cart 118 includes a pole 116 on which the container 114 hangs. Gravity causes the liquid in the container 114 to flow to the infusion pump 106. Once the liquid reaches the infusion pump 106, the infusion pump 106 controls the flow of the fluid for intravenous delivery of the fluid to a patient attachment site that can include a hypodermic needle that is mounted to a Luer fitting for delivery of the fluid to the venous system of the patient P.

The infusion pump 106 further includes a display 120 that can display parameters related to the delivery of the fluid to the venous system of the patient P. In some examples, the display 120 is a touchscreen that operates to receive tactile inputs from a user such as a caregiver C such that the display 120 is both a display device and a user input device.

The ventilator 108 controls delivery of oxygen to the patient P through tubing 122 which can include a first tube to deliver the oxygen to the patient P, and a second tube to take exhaled air away from the patient P. The ventilator 108 can include a humidifier 124 to warm and moisten the oxygen delivered to the patient P. The ventilator 108 is shown mounted on a mobile cart 126 such that the ventilator 108 is portable and can be brought into and out of the patient environment 100 as needed. The ventilator 108 further includes a display 128 that can display parameters related to the delivery of oxygen to the patient P. In some examples, the display 128 is a touchscreen that operates to receive tactile inputs from a user such as a caregiver C such that the display 128 is both a display device and a user input device.

As shown in FIG. 1, a caregiver C is shown located inside the patient environment 100. The caregiver C can use a mobile device 140 such as a smartphone, tablet computer, or other similar type of electronic device to enter physiological variable measurements that are measured by any of the medical devices inside the patient environment 100 such as the patient support apparatus 102, the patient monitoring device 104, the infusion pump 106, and the ventilator 108. The caregiver C can also use the mobile device 140 to enter clinical notes and observations. The physiological variable measurements, and clinical notes and observations entered on the mobile device 140 by the caregiver C can be stored in an electronic health record (EHR) 702 of the patient P that is maintained by an EHR system 300.

As described herein, the terms electronic medical records (EMRs) and electronic patient record (EPRs) can be used interchangeably with EHRs. The EHR system 300 collects patient electronically stored health information in a digital format (e.g., EHRs 302). As such, the EHR system 300 maintains a plurality of EHRs 302 for a plurality of patients admitted to the healthcare facility 10. Further, the EHRs 302 can be shared across multiple healthcare facilities through network-connected, enterprise-wide information systems or other information networks and exchanges. The EHRs 302 may include a range of data, including demographics, medical history, medication and allergies, immunization status, laboratory test results, radiology images, vital signs, personal statistics like age and weight, and billing information.

As shown in FIG. 1, a camera 200 that is mounted to a surface of the patient environment 100 such as a wall or ceiling. The camera 200 can be mounted at different locations within the patient environment 100 such that the mounting of the camera 200 as shown in FIG. 1 is provided by way of illustrative example. Alternatively, the healthcare facility 10 can include a plurality of cameras mounted onto multiple surfaces within the patient environment 100.

The camera 200 is configured to pan, tilt, and zoom for adjusting a view of the patient environment 100 as well as views of individual objects within the patient environment 100 such as the patient P, the patient support apparatus 102, the patient monitoring device 104, the infusion pump 106, the ventilator 108, and the caregiver C. The camera 200 can include a gimbal or similar structure actuated by an electric motor to pan the camera 200 left and right, and to tilt the camera 200 up and down. Also, the camera 200 can zoom in and out by adjusting a focal length of a lens whether mechanically (e.g., mechanical zoom) or digitally (e.g., digital zoom).

The video analytics system 500 receives from the camera 200 video data of the patient environment 100. As shown in FIG. 1, the video analytics system 500 is communicatively coupled to the camera 200 via a network 150. The network 150 connects and exchanges data between the camera 200 and the video analytics system 500, as well as between the camera 200 and other systems such as the EHR system 300. The network 150 can include any type of wired or wireless connections, or any combinations thereof. In some examples, the wireless connections can be accomplished using Wi-Fi, ultra-wideband (UWB), Bluetooth, and the like. In some examples, the network 150 is an Internet of things (IoT) network.

In some examples, the video analytics system 500 is communicatively connected to a workstation monitor 520 via the network 150. Alternatively, the video analytics system 500 can be connected directly to the workstation monitor 520 via wired and/or wireless connections without using the network 150. As will be described in more detail, the video analytics system 500 can display statuses of the medical devices and the caregivers C, and recommendations for allocations of the medical devices and the caregivers on the workstation monitor 520.

The video analytics system 500 extracts parameters from the video data captured by the camera 200 to determine a status such as a state of operation or functioning of the patient support apparatus 102, the patient monitoring device 104, the infusion pump 106, the ventilator 108, and other medical devices in the patient environment 100. For example, the video analytics system 500 can analyze the video data to determine whether the patient support apparatus 102 is occupied by the patient P, or is empty. Also, the video analytics system 500 can analyze the video data to determine whether the patient monitoring device 104, the infusion pump 106, the ventilator 108, and other medical devices positioned inside the patient environment 100 are turned on and are being used, are turned on and are running idle, or are turned off. The devices that are idle or turned off in the patient environment 100 can be counted or totalized for quantifying resources that are not being used in the patient environment. As another example, the video analytics system 500 can analyze the video data to determine consumption of consumables such as disposable temperature probe covers used by the patient monitoring device 104.

The video analytics system 500 can also analyze the video data captured by the camera 200 to determine operational metrics such as how long it took a caregiver C to setup or interact with a medical device. In some examples, the operational metrics include identification of users of the medical devices, classification of the users into one or more categories or classes, and interactions by users with the medical devices in a clickstream-like analysis. For example, the video analytics system 500 can identify which features of the medical devices are used by the users, and which features of the medical devices are not used by the users. Further, the video analytics system 500 can identify an order in which the features of the medical devices are used. Such operational metrics can be associated with the model numbers of the medical devices to improve caregiver training to more efficiently and/or effectively use the medical devices.

Examples of the users identified in the clickstream-like analysis of the video data can include caregivers and other clinicians as well as patients. The users can also be grouped by department, unit, floor, or other environment in the healthcare facility 10. Further, the operational metrics determined from the analysis can be categorized by disease state.

Examples of the data collected in the clickstream-like analysis of the video data can include user metadata and/or profile, medical device features used, type of care environment (e.g., operating room versus med-surge room) as well as whether a medical device is used alone by itself, or with other types of medical devices. Further examples of the data collected can include how the medical devices are used such as whether the medical devices are used under one or more types of configurations or modes of operation. Further examples of the data collected can include downstream key performance indicators (KPIs) such as patient readmission rate, sentinel event (e.g., code blue), dropout rate, consumables used, and idle resource time.

The video analytics system 500 can adjust operations of the medical devices inside the patient environment 100 based on their statuses such that the medical devices are used more efficiently in the healthcare facility 10. Further, the video analytics system 500 can display recommendations on the workstation monitor 520 for adjusting allocations and/or usage of the medical devices within the healthcare facility 10. Further, the video analytics system 500 can display benchmark comparisons on the workstation monitor 520 related to the usage and/or allocations of the medical devices in the healthcare facility 10 with benchmark metrics determined from other healthcare facilities that share one or more similarities with the healthcare facility 10 such as size, medical specialty or focus, geographic location, and the like.

Additionally, the video analytics system 500 extracts parameters from the video data captured by the camera 200 to classify caregivers C who enter and exit the patient environment 100. For example, the video analytics system 500 can classify the caregivers C as physicians, registered nurses, medical technicians, and the like. The video analytics system 500 can classify the caregivers C based on articles worn by the caregivers such as clothing, instruments carried by the caregivers (e.g., stethoscope), and other features identifiable from the video data.

The video analytics system 500 can adjust operations of the medical devices inside the patient environment 100 based on a classification of the caregiver C such that the medical devices are used more efficiently in the healthcare facility 10. Further, the video analytics system 500 can display recommendations on the workstation monitor 520 for adjusting allocations of caregivers within the healthcare facility 10 based on their classification. Further, the video analytics system 500 can display benchmark comparisons on the workstation monitor 520 related to the allocations of the caregivers in the healthcare facility 10 with benchmark metrics determined from other healthcare facilities that share one or more similarities with the healthcare facility 10 such as size, medical specialty or focus, geographic location, and the like.

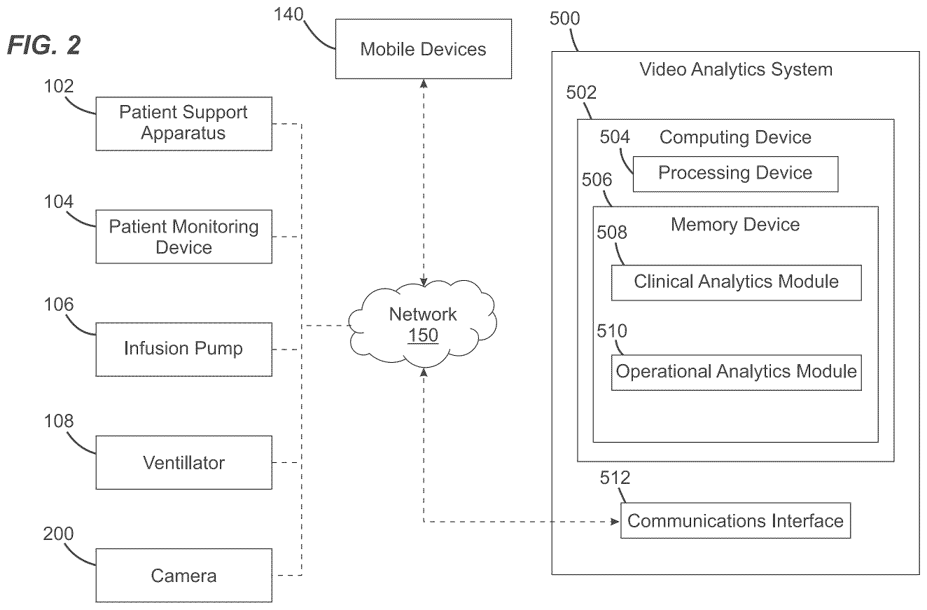

FIG. 2 schematically illustrates an example of the video analytics system 500 communicatively coupled via the network 150 to the patient support apparatus 102, the patient monitoring device 104, the infusion pump 106, the ventilator 108, the camera 200, and the mobile devices 140 carried by the caregivers C. The video analytics system 500 includes a communications interface 512 that allows the video analytics system 500 to connect to the network 150. The communications interface 512 can include wired interfaces and wireless interfaces. For example, the communications interface 512 can wirelessly connect to the network 150 through cellular network communications, Wi-Fi, and other wireless connections. Alternatively, the communications interface 512 can connect to the network 150 using wired connections such as through an Ethernet or Universal Serial Bus (USB) cable.

The video analytics system 500 includes a computing device 502 having at least one processing device 504 and at least one memory device 506. The at least one processing device 504 is an example of a processing unit such as a central processing unit (CPU). The at least one processing device 504 can include one or more central processing units (CPUs). In some examples, the at least one processing device 504 includes one or more digital signal processors, field-programmable gate arrays, and/or other types of electronic circuits.

The at least one memory device 506 is an example of a computer readable data storage device that operates to store data and instructions for execution by the at least one processing device 504. For example, the at least one memory device 506 stores a clinical analytics module 508 and an operational analytics module 510, which are described in more detail below. The at least one memory device 506 includes computer-readable media, which includes any media that can be accessed by the at least one processing device 504.

By way of example, computer-readable media include computer readable storage media and computer readable communication media. Computer readable storage media includes volatile and nonvolatile, removable and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the data acquisition device. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

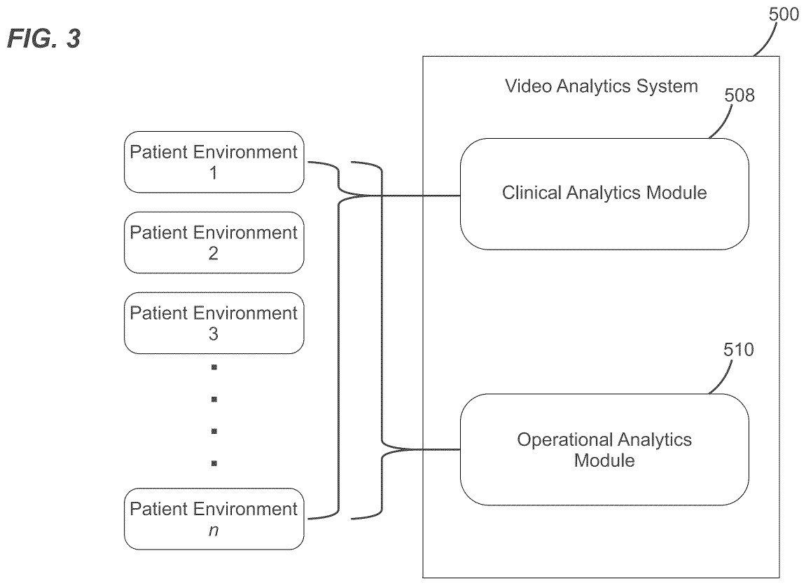

FIG. 3 schematically illustrates an example of the clinical analytics module 508 and the operational analytics module 510 implemented on the video analytics system 500. The video analytics system 500 receives video data from a plurality of patient environments 100 (i.e., patient environment 1 . . . patient environment n) in the healthcare facility 10.

The clinical analytics module 508 analyzes the video data to optimize use of the medical devices in the patient environments 100. For example, the clinical analytics module 508 can optimize use of the medical devices based on their status detected from the video data. Also, the clinical analytics module 508 can optimize use of the medical devices based on a classification of a caregiver in proximity to the medical devices detected from the video data.

The clinical analytics module 508 can further provide patient safety monitoring by detecting one or more events such as when a patient P attempts to exit the patient support apparatus 102 without assistance to reduce patient falls risk; recognizing when a patient P is in distress or showing signs of medical complications such as seizures; and monitoring for any unplanned removal of devices, equipment, and/or instruments such as intravenous (IV) lines from the infusion pump 106, oxygen masks and/or the tubing 122 of the ventilator 108, and the like. When such events are detected, the clinical analytics module 508 generates an alert for display on the mobile devices 140 and/or on the workstation monitor 520.

The clinical analytics module 508 can further provide environmental safety monitoring such as detecting obstacles such as the IV lines from the infusion pump 106 and/or the tubing 122 of the ventilator 108 that are trip hazards. The environmental safety monitoring can further include monitoring whether medical devices such as the patient monitoring device 104, the infusion pump 106, and the ventilator 108 are returned to their proper storage location when the devices are turned off and/or are no longer being used. The environmental safety monitoring can further include monitoring conditions of the patient environment 100 such as spills, lighting, temperature, air quality, and the like.

When an environmental safety hazard is detected, the clinical analytics module 508 can adjust an operation of a medical device to alert a caregiver C of the safety hazard. For example, the clinical analytics module 508 can display a notification on the display 120 of the infusion pump 106 to alert a caregiver that the IV lines are a trip hazard. As another example, the clinical analytics module 508 can display a notification on the display 128 of the infusion pump 106 to alert a caregiver that the tubing 122 is a trip hazard. As another example, the clinical analytics module 508 can display a notification on the display 110 of the patient monitoring device 104 to alert a caregiver that the patient monitoring device 104 is blocking a pathway or an exit/entrance of the patient environment 100 when the patient monitoring device 104 is not returned to its proper storage location when not being used.

The clinical analytics module 508 can further monitor treatment and/or recovery progress such as monitoring for wound healing, signs of infection or complications, monitoring patient's physical movements for physiotherapy progress, and detecting early signs of complications such as swelling or discoloration around a surgical site on a patient P.

The clinical analytics module 508 can provide a number of technical advantages such as enhanced patient safety because risks and safety hazards are quickly detected and addressed to reduce adverse events. Further, the clinical analytics module 508 can improve efficiency in the healthcare facility 10 by providing real-time feedback, allowing caregivers C to immediately address issues or needs of the patients P inside the patient environments 100. Further, the clinical analytics module 508 can reduce human errors by automating the monitoring process and providing an additional layer of oversight.

The operational analytics module 510 can include a set of algorithms that analyze the video data captured from the patient environments 100 to measure metrics related to the healthcare provided to the patients P in the healthcare facility 10. The metrics are automatically measured by the operational analytics module 510, and are measured in real-time, at the point of care (e.g., the patient environment 100). Thus, the metrics are not based on charted data or information that is reported periodically (e.g., quarterly). Also, the metrics are automatically updates as the dynamics of the patient environment 100 vary. Accordingly, the operational analytics module 510 captures and analyzes the video data dynamically to measure metrics related to the healthcare provided to the patients P in the healthcare facility 10 without requiring user input such that user errors and/or omissions are eliminated.

The metrics measured by the operational analytics module 510 can include caregiver interaction monitoring such as tracking caregiver-patient interactions to ensure that check-ins and procedures occur on schedule, monitoring hand hygiene adherence among the caregivers C and other healthcare workers, and ensuring the patients P receive correct treatments. The operational analytics module 510 can enhance accountability by ensuring protocols set by the healthcare facility 10 or other organizations are followed to improve healthcare quality.

The operational analytics module 510 can further include a set of algorithms that analyze the video data captured from the patient environments 100 to measure metrics related to the use and allocation of resources in the healthcare facility 10. The use and allocation of resources include the allocation and usage of the medical devices in the plurality of patient environments 100. The use and allocation of resources can further include the allocation of personnel such as the caregivers C among the plurality of patient environments 100.

The operational analytics module 510 allows the healthcare facility 10 to capture, monitor, allocate, and forecast the use and allocation of resources for improving operational planning for the healthcare facility 10. For example, the metrics captured by the operational analytics module 510 can provide comprehensive operations information.

As an illustrative example, the metrics captured by the operational analytics module 510 can be relevant to financial accounting and reporting such as expenses from caregiver salaries, consumption of supplies, medical device usage, and other operational costs. As a further example, the metrics captured by the operational analytics module 510 can be relevant to budgeting and forecasting such as for purchasing and/or leasing new medical devices and equipment, facility expansions and capital improvements, and forecasting day-to-day expenses in the healthcare facility 10. As a further example, the metrics captured by the operational analytics module 510 can be relevant to patient data and analytics such as length of stay (LOS) including average durations that the patients P stay in the healthcare facility 10, which is a significant factor in resource utilization. The metrics captured by the operational analytics module 510 can be further relevant to complication rates from surgeries and treatments, and patient satisfaction relevant to the quality of healthcare and overall experience.

The metrics captured by the operational analytics module 510 can include operational metrics such as bed occupancy rates for effective capacity management; wait times for room admission, treatments, and food delivery; staff-to-patient ratios to ensure adequate staffing; and turnover rates for both staff and beds, which can indicate efficiency. The metrics captured by the operational analytics module 510 can further include measuring visits and time present of the caregivers C inside the patient environments 100.

The metrics captured by the operational analytics module 510 can be relevant to human resource management such as staffing levels across different caregiver classifications and departments, units, floors, and the like within the healthcare facility 10, recruitment and retention data to gauge employee satisfaction and identify areas of improvement, and caregiver training and development by recording ongoing education and certification efforts.

The metrics captured by the operational analytics module 510 can be relevant to supply chain and inventory such as usage/consumption rates for consumables used by the medical devices which can be used to estimate order frequencies and volumes to optimize procurement strategies, and adjust vendor contracts for cost-saving opportunities.

The metrics captured by the operational analytics module 510 can also be relevant to compliance and regulatory reporting such as for accreditation status to ensure the healthcare facility 10 meets regional and national standards; for generating audit reports, both financial and clinical, to assess risk areas; and for generating regulatory filings.

The metrics captured by the operational analytics module 510 can also be relevant to strategic planning and market analysis including competitive analyses provision insights into how the healthcare facility 10 compares to competing healthcare facilities; market trends based on changing healthcare needs, technological advancements, or regional population shifts; and growth opportunities by identifying potential areas for expansion or new service offerings.

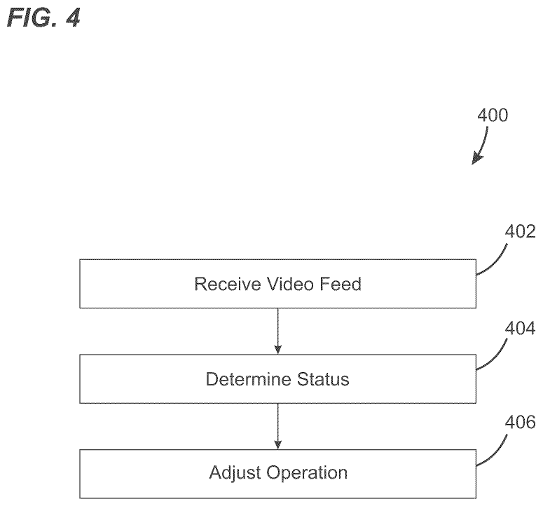

FIG. 4 schematically illustrates an example of a method 400 of improving medical device performance in the healthcare facility 10. At least certain aspects of the method 400 can be performed by the video analytics system 500.

The method 400 includes an operation 402 of receiving video data of a patient environment 100 where at least one medical device is positioned. The video data can be received by the video analytics system 500 from the camera 200 via the network 150. The at least one medical device can include at least one of the patient support apparatus 102, the patient monitoring device 104, the infusion pump 106, and the ventilator 108.

The method 400 includes an operation 404 of analyzing the video data to determine a status of the at least one medical device. Operation 404 can include determining an error status of the at least one medical device based on the analysis of the video data. The error status can include on observation from the video data that the at least one medical device is not being effectively use or is malfunctioning. As an illustrative example, an error status is determined based on the video data when an IV line of the infusion pump 106 is removed from the patient. As another illustrative example, an error status is determined based on the video data when the tubing 122 from the ventilator 108 is disconnected either from the ventilator or from the patient.

The error status can also be based on an observation from the video data that the at least one medical device is a risk hazard such as when the IV line of the infusion pump 106 or the tubing 122 of the ventilator 108 presents a tripping hazard inside the patient environment 100, or when portable medical devices such as the patient monitoring device 104, the infusion pump 106, and the ventilator 108 are blocking an exit or entry of the patient environment 100.

Operation 404 can further include determining an idle status of the at least one medical device based on the analysis of the video data. The idle status indicates that the at least one medical device is turned on, but is not being used inside the patient environment 100.

Operation 404 can further include determining a shortage status of the at least one medical device based on the analysis of the video data. The shortage status indicates a low supply of a consumable that is used by the at least one medical device. As an illustrative example, the shortage status can relate to a remaining quantity or supply of disposable temperature probe covers used by the patient monitoring device 104.

The method 400 includes an operation 406 of adjusting an operation of the at least one medical device based on the status determined in operation 404. For example, when operation 404 determines an error status of the at least one medical device, operation 406 can include altering a graphical user interface on a display of the at least one medical device such as the display of the patient support apparatus 102, the display 110 on the patient monitoring device 104, the display 120 on the infusion pump 106, or the display 128 on the ventilator 108. The graphical user interface can be altered to display an alert or warning that the at least one medical device is not being effectively use or is malfunctioning. For example, an alert or warning can be displayed on the display 120 of the infusion pump 106 to indicate that the IV line is removed from the patient. As another example, an alert or warning can be displayed on the display 128 of the ventilator 108 to indicate that the tubing 122 of the ventilator 108 is disconnected. As a further example, alerts or warnings can be displayed on the respective displays of the patient monitoring device 104, the infusion pump 106, and the ventilator 108 when accessories of these portable medical devices are a tripping hazard, ow when these portable medical devices are blocking an exit or entry of the patient environment 100.

When operation 404 determines an idle status of the at least one medical device, operation 406 can include turning the at least one medical device off to conserve energy consumption by the medical device. Further, by turning off the medical device, the lifetime of the medical device can be extended because idling medical devices are often exposed to contamination and experience wear and tear, and as a result can be damaged when idling for extended periods of time inside the patient environment 100.

When operation 404 determines a shortage status of the at least one medical device, operation 406 can include adjusting the operation of the at least one medical device to consume less consumables. In further examples, when operation 404 determines a shortage status of the at least one medical device, operation 406 can include altering a graphical user interface on a display of the at least one medical device to alert a user of the shortage of the consumable such as the disposable temperature probe covers used by the patient monitoring device 104.

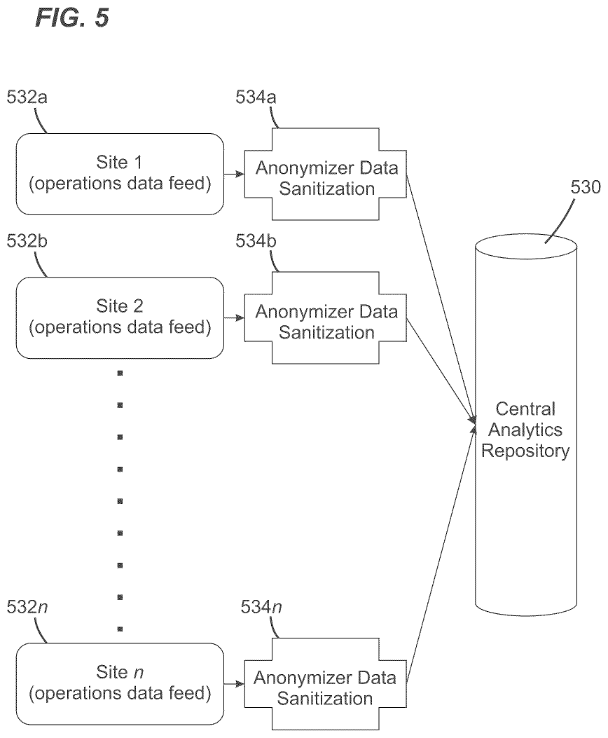

FIG. 5 schematically illustrates an example of a central analytics repository 530 that can be managed by the video analytics system 500. The central analytics repository 530 provides cross-institutional data aggregation. The central analytics repository 530 receives video data feeds 532a-532n from a plurality of healthcare facilities 10a-10n. As an illustrative example, the plurality of healthcare facilities 10a-10n can agree to share the video data feeds 532a-532n with the central analytics repository 530 via a bi-lateral data sharing agreement. Each video data feed 532a-532n includes video data captured from a plurality of patient environments 100 within a healthcare facility. As shown in FIG. 5, the video data feeds 532a-532n are converted into anonymized data 534a-534n for storage in the central analytics repository 530. As will now be described in more detail, the central analytics repository 530 uses the anonymized data 534a-524n to provide benchmarking for metrics determined for the healthcare facility 10.

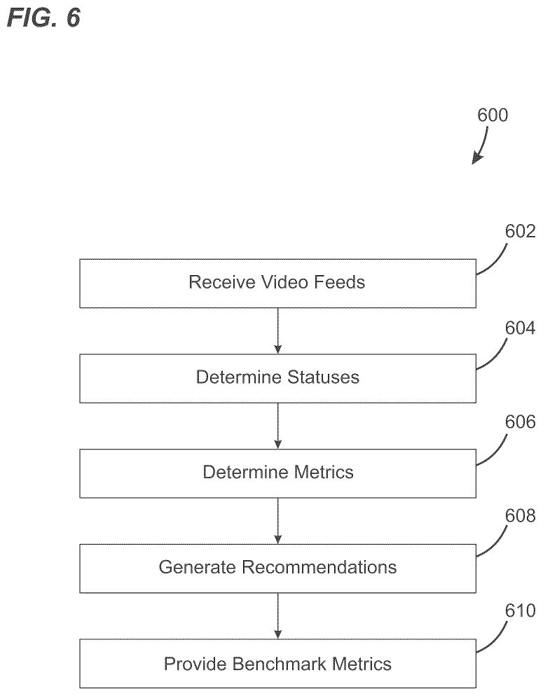

FIG. 6 schematically illustrates an example of a method 600 of benchmarking metrics determined for the healthcare facility 10. Aspects of the method 600 that can be performed by the video analytics system 500. As described above, the healthcare facility 10 can include a plurality of patient environments 100 each containing a camera 200 and one or more medical devices for providing healthcare to one or more patients in the patient environments.

The method 600 includes an operation 602 of receiving video data of a plurality of patient environments 100 within the healthcare facility 10. The video data of the plurality of patient environments is captured by at least one camera 200 in each of the plurality of patient environments 100. The video analytics system 500 can receive the video data of the plurality of patient environments 100 from the cameras 200 over the network 150.

The method 600 includes an operation 604 of analyzing the video data of the plurality of patient environments 100 to determine statuses of a plurality of medical devices in the healthcare facility 10. For example, operation 604 can include determining statuses of a plurality of the patient support apparatuses 102, a plurality of the patient monitoring devices 104, a plurality of the infusion pumps 106, and a plurality of the ventilators 108.

The method 600 includes an operation 606 of determining metrics for the healthcare facility 10 such as based on the statuses determined in operation 604 for the plurality of medical devices in the healthcare facility 10. For example, operation 606 can include determining metrics for the healthcare facility 10 based on the statuses of the plurality of the patient support apparatuses 102, the statuses of the plurality of the patient monitoring devices 104, the statuses of the plurality of the infusion pumps 106, and the statuses of the plurality of the ventilators 108.

The method 600 includes an operation 608 of generating recommendations based on the metrics determined in operation 606. As an illustrative example, operation 608 can include generating a recommendation to reallocate the plurality of medical devices among the plurality of patient environments 100 within the healthcare facility 10 based on the statuses of the plurality of medical devices. When operation 606 determines that a large percentage of a certain type of medical device such as the ventilators 108 are running idle or are turned off based on their statuses determined in operation 604, operation 608 can include recommending reallocation of the certain type of medical device (e.g., ventilators 108) to another department, unit, or floor of the healthcare facility 10 where there is an existing demand for using the medical devices.

In some instances, operation 608 can further include adjusting the operation of at least one medical device based on the metrics determined from the statuses of the plurality of medical devices. As an illustrative example, operation 608 can include turning off a certain type of medical device when the metrics determined in operation 606 identify a large percentage of the type of medical device are running idle in the healthcare facility 10. As discussed above, this can reduce energy consumption and extend the lifetime of the medical devices.

The method 600 includes an operation 610 of providing benchmark metrics for comparison with the metrics determined in operation 606. As discussed above in view of FIG. 5, the benchmark metrics are generated from the anonymized data 534a-524n, which is generated from video data feed 532a-532n received from a plurality of healthcare facilities 10a-10n. The comparison of the metrics determined in operation 606 with the benchmark metrics provided in operation 610 can be displayed on the workstation monitor 520.

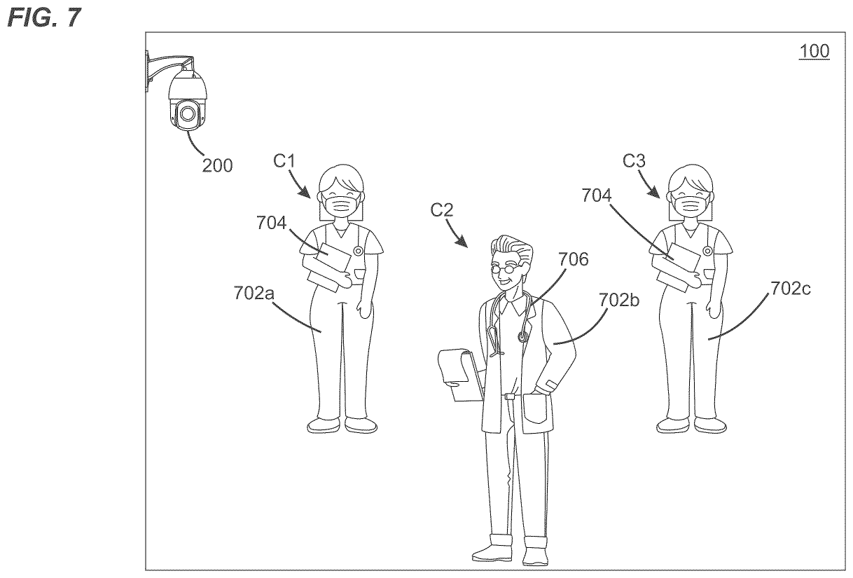

FIG. 7 illustrates examples of caregivers C that can be classified by the video analytics system 500 based on their attire, one or more objects carried by the caregivers, or other identifiable features that can be detected by video. The caregivers C are shown as being located inside a patient environment 100 that includes a camera 200 that captures video data of the patient environment 100. As described above, the patient environment 100 can be an operating room, a med-surg patient room, or other type of patient room within the healthcare facility 10.

In the illustrative example of FIG. 7, a first type of caregiver C1 is wearing a first type of garment 702a, a second type of caregiver C2 is wearing a second type of garment 702b, and a third type of caregiver C3 is wearing a third type of garment 702c. The first, second, and third types of garments 702a-702c are distinguishable from one another in one or more aspects. For example, the first, second, and third types of garments 702a-702c can have different colors, patterns, or styles. The first, second, and third types of garments 702a-702c are shown for illustrative purposes and it is contemplated that additional types of garments or fewer types of garments may be worn by the plurality of caregivers C within the healthcare facility.

As further shown in FIG. 7, the caregivers C1-C3 are carrying different objects. For example, the first and third caregivers C1, C3 are shown holding a patient chart 704, while the second caregiver C2 is shown as having a stethoscope 706 placed around their neck. As will now be described in more detail, the video analytics system 500 uses the video data captured from the camera 200 to detect one or more aspects of the garments and objects carried by the caregivers C1-C3 to classify the caregivers C1-C3 into one or more categories of caregivers.

The video analytics system 500 classifies the caregivers C1-C3 based on the garments 702 worn by the caregivers. For example, the second type of garment 702b (e.g., lab coat) worn by the caregiver C2 distinguishes the caregiver from the first and third types of caregivers C1, C3 who wear a different type or style of clothing (e.g., scrubs). Also, the video analytics system 500 classifies the caregivers C1-C3 based on a color of the clothing worn by the caregivers. For example, the healthcare facility 10 can have a clothing color convention to distinguish different types of caregivers such that the first type of caregiver C1 (e.g., registered nurses) may wear scrubs having a first color (e.g., blue) and the third type of caregiver C3 (e.g., a medical technician) may wear scrubs having a second color (e.g., green).

The video analytics system 500 can adjust operations of the medical devices inside the patient environment 100 based on a classification of a caregiver in the patient environment 100 such that the medical devices are configurable for more efficient and effective use by a caregiver C. For example, the video analytics system 500 can adjust the graphical user interfaces displayed on the displays of the medical devices based on the classification of the caregiver C.

As an illustrative example, the video analytics system 500 can adjust the graphical user interfaces displayed on the displays of the medical devices to disable features when the caregiver is classified as a caregiver having a lower skill set such that the graphical user interfaces displayed on the displays of the medical devices are simplified and easier to use by the caregiver. Alternatively, the video analytics system 500 can adjust the graphical user interfaces displayed on the displays of the medical devices to enable features when the caregiver is classified as a caregiver having a higher skill set to provide additional features for selection by the caregiver for more effective use of the medical devices by the caregiver.

As another example, the video analytics system 500 can automatically silence an alarm triggered on a medical device in the patient environment 100 when based on the video data captured by the camera 200, the video analytics system 500 determines that a caregiver C has entered the patient environment 100, and the video analytics system 500 classifies the caregiver as having a skillset such that the caregiver is able to address a condition that triggered the alarm. Advantageously, this can mitigate alarm fatigue within the healthcare facility 10.

Further, the video analytics system 500 can display recommendations on the workstation monitor 520 for adjusting allocations of the caregivers within the healthcare facility 10. For example, the video analytics system 500 can determine visit frequencies and time spent by certain classes of caregivers in the patient environments within a unit or department of the healthcare facility 10, and the video analytics system 500 can recommend allocating additional caregivers or allocating fewer caregivers to the unit or department based on the determined visit frequencies and time spent in the patient environments of the unit or department. Additionally, the video analytics system can analyze the video data to distinguish a patient's delayed return to the patient environment 100 from an abscondence even, and can count a quantity of delayed return events versus a quantity of abscondence events for admission/discharge metrics.

Further, the video analytics system 500 can display benchmark comparisons on the workstation monitor 520 related to the allocations of the caregivers in the healthcare facility 10 with benchmark metrics determined from other healthcare facilities that share one or more similarities with the healthcare facility 10 such as size, medical specialty or focus, geographic location, and the like. The video analytics system 500 can compare visit frequencies or time spent in the patient environments by physicians, registered nurses, medical technicians, and other classifications of caregivers with benchmark metrics for these classifications of caregivers determined from the other healthcare facilities that share the one or more similarities.

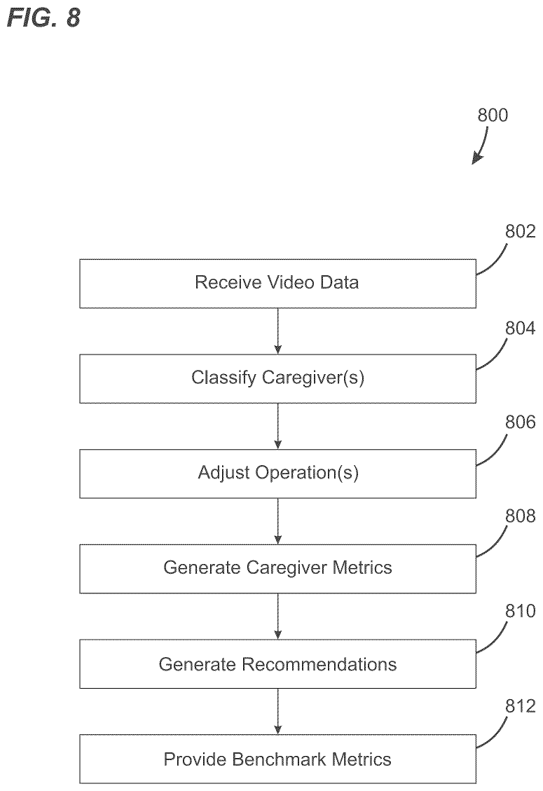

FIG. 8 schematically illustrates an example of a method 800 of improving healthcare provided in the healthcare facility 10. Aspects of the method 800 can be performed by the video analytics system 500. As shown in FIG. 8, the method 800 includes an operation 802 of receiving video data from a camera 200 inside a patient environment 100. The video data can be received by the video analytics system 500 from the camera 200 via the network 150.

The method 800 includes an operation 804 of classifying one or more caregivers in the video data received in operation 802. Operation 804 can include classifying the one or more caregivers based on one or more aspects of the garments worn by the caregivers such as a color, a pattern, or a style of the garments. Operation 804 can also include classifying the one or more caregivers based on one or more objects carried, held, or worn by the caregivers such as a patient chart 704 or a stethoscope 706, as shown in the example provided in FIG. 7. The video data captured by the camera 200 is analyzed to identify the one or more caregivers C inside the patient environment 100, as well as to detect the one or more aspects of the garments worn by the caregivers C and the objects carried by the caregivers C. In some instances, machine learning or other type of artificial intelligence is used to identify the caregivers C, and to detect the one or more aspects of the garments and the objects carried by one or more caregivers C.

The method 800 can include an operation 806 of adjusting operations of at least one medical device inside the patient environment 100 based on the classification of a caregiver determined in operation 804. As an illustrative example, operation 806 can include adjusting a graphical user interface on a display of the at least one medical device such as the display of the patient support apparatus 102, the display 110 on the patient monitoring device 104, the display 120 on the infusion pump 106, or the display 128 on the ventilator 108. For example, the graphical user interface can be adjusted to display a predetermined set of controls based on the classification of the caregiver determined in operation 804. The predetermined set of controls may disable certain features when the caregiver is classified as a caregiver having a lower skill set such that the graphical user interfaces displayed on the displays of the medical devices are simplified and easier to use by the caregiver for more efficient use of the medical devices by the caregiver. Alternatively, the predetermined set of controls may enable features when the caregiver is classified as a caregiver having a higher skill set to provide additional features for selection by the caregiver for more effective use of the medical devices by the caregiver.

The method 800 can further include an operation 808 of generating caregiver metrics based on the classifications of the caregivers. For example, the caregiver metrics can be determined from monitoring the video data captured by the camera 200 over a period of time to determine classifications of caregivers who enter and exit the patient environment 100. Operation 808 can include determining visit frequencies and time spent by different classifications of caregivers in one or more patient environments 100.

The caregiver metrics generated in operation 808 can be used to track patient care. As an illustrative example, operation 808 can include monitoring hourly patient assessments by registered nurses (RNs) for a department, a unit, or a floor within the healthcare facility 10, or the healthcare facility 10 overall. As another example, operation 808 can include monitoring Q4 vitals by medical technicians for a department, a unit, or a floor within the healthcare facility 10, or the healthcare facility 10 overall. As another example, operation 808 can include measuring response times for one or more classes of caregivers to alarms and nurse calls for a department, a unit, or a floor within the healthcare facility 10, or the healthcare facility 10 overall. The metrics generated in the operation 808 can be used to identify areas for improvement in patient care.

The method 800 can further include an operation 810 of generating recommendations based on the caregiver metrics generated in operation 808. For example, operation 810 can include generating a recommendation to adjust an allocation of caregivers within the healthcare facility 10 such as to allocate additional caregivers to a department, a unit, or a floor of the healthcare facility based on the determined visit frequencies and time spent in the patient environments of the department, the unit, or the floor. Alternatively, operation 810 can include generating a recommendation to allocate fewer caregivers to a department, a unit, or a floor of the healthcare facility based on the determined visit frequencies and time spent in the patient environments of the department, the unit, or the floor.

The method 800 can further include an operation 812 of providing benchmark metrics for comparison with the caregiver metrics determined in operation 808. The benchmark metrics are generated from the anonymized data 534a-524n, which is generated from video data feed 532a-532n received from a plurality of healthcare facilities 10a-10n that share one or more similarities with the healthcare facility 10 such as size, medical specialty or focus, geographic location, and the like. The caregiver metrics determined in operation 808 can be compared to the benchmark metrics such as to compare visit frequencies or time spent in the patient environments by physicians, registered nurses, medical technicians, and other classifications of caregivers.

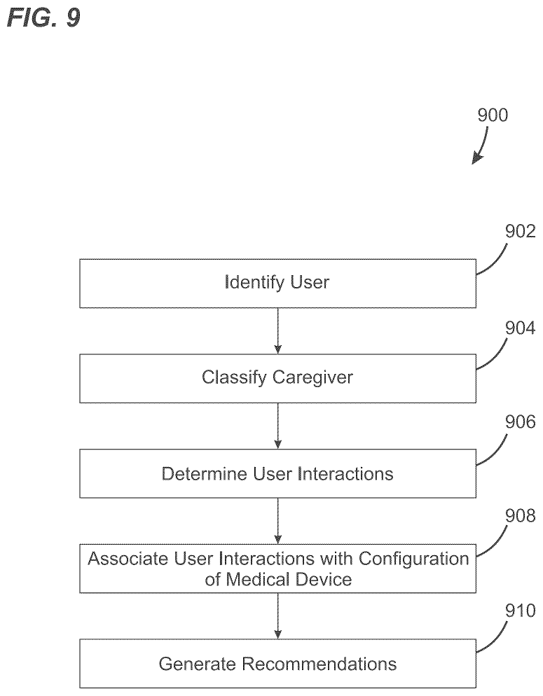

FIG. 9 schematically illustrates an example of a method 900 of improving usage of the medical devices in the healthcare facility 10. Aspects of the method 900 can be performed by the video analytics system 500. The method 900 includes an operation 902 of identifying a user who enters the patient environment 100. Operation 902 can include using machine learning or other artificial intelligence algorithms to identify the user.

The method 900 includes an operation 904 of classifying the user into one or more categories or classes. For example, operation 904 can include classifying the user as a physician, registered nurse, medical technician, or the like. Operation 904 can further include classifying the user as a patient or a family member who is not a trained medical professional. Operation 904 can include classifying the user based on one or more aspects of a garment worn by the user such as color, pattern, or style, and/or based on one or more objects carried by the user, as described above. Operation 904 can include using machine learning or other artificial intelligence algorithms to classify the user into the one or more categories or classes.

The method 900 includes an operation 906 of determining interactions of the user with a medical device such as the patient support apparatus 102, the patient monitoring device 104, the infusion pump 106, and the ventilator 108. Operation 906 can include determining which features of the medical device are used and in what order. Operation 906 can further include measuring an amount of time interacting with the medical device.

The method 900 includes an operation 908 of associating the user interactions determined in operation 906 with a configuration of the medical device. For example, operation 908 can include associating the user interactions with a model number of the medical device. In further examples, operation 908 can include associating the user interactions with a configuration or mode of operation of the medical device. In further examples, operation 908 can include associating the user interactions with an environment in which the medical device is used.

The method 900 includes an operation 910 of generating recommendations for more efficiently or effectively using the medical device. In some instances, the recommendations are generated based on aggregated data collected from a plurality of interactions by different users with the medical device. The recommendations can be displayed on the workstation monitor 520, or can be displayed on a display of the medical device such as the display of the patient support apparatus 102, the display 110 on the patient monitoring device 104, the display 120 on the infusion pump 106, or the display 128 on the ventilator 108. The recommendations can include using one or more features of the medical device that were not used, or using the one or more features of the medical device in a different order. Such recommendations can improve the performance of the medical device such as by ensuring that all the features of the medical device are used and/or that the time spent setting-up or interacting with the medical device is reduced.

As another example, operation 910 can include generating an alert to remind a user to perform a task that has not yet been completed after a predetermined amount of time has elapsed since the user's interaction with the medical device. For example, operation 910 can include sending a message to a caregiver identified in operation 902 to prompt the caregiver to enter data captured by the medial device into the EHR 302 of the patient after the predetermined amount of time (e.g., 30 minutes) has elapsed since the caregiver's last interaction with the medical device.

In some examples, the recommendations generated in operation 910 are implemented on the medical device. For example, the user-medical device interactions determined in operation 906 can identify a sequence of actions which indicates confusion on how to effectively use the medical device. In such instances, the user interface displayed on the medical device (e.g., the display of the patient support apparatus 102, the display 110 on the patient monitoring device 104, the display 120 on the infusion pump 106, or the display 128 on the ventilator 108) is updated such that the user interface is more easily navigable to reach a desired tool or feature of the medical device more quickly and efficiently.

The video analytics system 500 and the methods described herein provide several technical advantages over real-time location systems (RTLS) because the video analytics system 500 and the methods described herein eliminate the need for the caregivers to carry physical tracking devices which require physical management, service, and back-end infrastructure. Instead, the video analytics system 500 and the methods described herein passively monitor the medical devices, caregivers, and caregiver-medical device interactions such that the monitoring is less cumbersome on the caregivers, and eliminates costs related to purchasing and maintaining physical tracking devices within the healthcare facility 10. Further, detailed metrics such as caregiver-medical device interactions including which features are used and in what order can be extracted from the video data that are not possible to extract from location tracking data.

The various embodiments described above are provided by way of illustration only and should not be construed to be limiting in any way. Various modifications can be made to the embodiments described above without departing from the true spirit and scope of the disclosure.

What is claimed is:

1. A system for improving medical device performance, the system comprising:
at least one processing device; and
at least one computer readable data storage device storing software instructions that, when executed by the at least one processing device, cause the at least one processing device to:
receive video data of a patient environment where at least one medical device is positioned;
analyze the video data to determine a status of the at least one medical device; and
adjust an operation of the at least one medical device based on the status.

2. The system of claim 1, wherein the instructions, when executed by the at least one processing device, further cause the at least one processing device to:
receive video data of a plurality of patient environments within a healthcare facility;
analyze the video data of the plurality of patient environments to determine statuses of a plurality of medical devices in the healthcare facility; and
adjust the operations of the plurality of medical devices based on the statuses of the plurality of medical devices.

3. The system of claim 2, wherein the instructions, when executed by the at least one processing device, further cause the at least one processing device to:
generate a recommendation to reallocate the plurality of medical devices among the plurality of patient environments within the healthcare facility based on the statuses of the plurality of medical devices.

4. The system of claim 2, wherein the instructions, when executed by the at least one processing device, further cause the at least one processing device to:
generate benchmark metrics for comparing the healthcare facility to other healthcare facilities based on the statuses of the plurality of medical devices.

5. The system of claim 1, wherein when analysis of the video data determines an error status of the at least one medical device, adjust the operation of the at least one medical device includes altering a graphical user interface on a display of the at least one medical device.

6. The system of claim 1, wherein when analysis of the video data determines an idle status of the at least one medical device, adjust the operation of the at least one medical device includes turning off the at least one medical device.

7. The system of claim 1, wherein when analysis of the video data determines a shortage status of the at least one medical device, adjust the operation of the at least one medical device includes adjusting the operation of the at least one medical device to consume less consumables.

8. The system of claim 1, wherein the instructions, when executed by the at least one processing device, further cause the at least one processing device to:
analyze the video data to determine a classification of a caregiver inside the patient environment; and
adjust the operation of the at least one medical device based on the classification of the caregiver inside the patient environment.

9. The system of claim 1, wherein the instructions, when executed by the at least one processing device, further cause the at least one processing device to:
monitor the video data over a period of time to determine classifications of a plurality of caregivers who enter and exit the patient environment over the period of time; and
generate metrics based on the classifications of the plurality of caregivers.

10. The system of claim 1, wherein the instructions, when executed by the at least one processing device, further cause the at least one processing device to:
generate a recommendation to adjust an allocation of caregivers assigned to the patient environment based on at least one of a visit frequency and a time spent by the caregivers in the patient environment determined from the video data of the patient environment.

11. A method of improving medical device performance, the method comprising:
receiving video data of a patient environment where at least one medical device is positioned;
analyzing the video data to determine a status of the at least one medical device; and
adjusting an operation of the at least one medical device based on the status.

12. The method of claim 11, further comprising:
receiving video data of a plurality of patient environments within a healthcare facility;
analyzing the video data of the plurality of patient environments to determine statuses of a plurality of medical devices in the healthcare facility; and
adjusting the operations of the plurality of medical devices based on the statuses of the plurality of medical devices.

13. The method of claim 12, further comprising:
generating a recommendation to reallocate the plurality of medical devices among the plurality of patient environments within the healthcare facility based on the statuses of the plurality of medical devices.

14. The method of claim 12, further comprising:
generating benchmark metrics for comparing the healthcare facility to other healthcare facilities based on the statuses of the plurality of medical devices.

15. The method of claim 11, wherein when analyzing the video data determines an error status of the at least one medical device, adjusting the operation of the at least one medical device includes altering a graphical user interface on a display of the at least one medical device.

16. The method of claim 11, wherein when analyzing the video data determines an idle status of the at least one medical device, adjusting the operation of the at least one medical device includes turning off the at least one medical device.

17. The method of claim 11, wherein when analyzing the video data determines a shortage status of the at least one medical device, adjusting the operation of the at least one medical device includes adjusting the operation of the at least one medical device to consume less consumables.

18. The method of claim 11, further comprising:
analyzing the video data to determine a classification of a caregiver inside the patient environment; and
adjusting the operation of the at least one medical device based on the classification of the caregiver inside the patient environment.

19. The method of claim 11, further comprising:
monitoring the video data over a period of time to determine classifications of a plurality of caregivers who enter and exit the patient environment over the period of time; and
generating metrics based on the classifications of the plurality of caregivers.

20. A system for improving medical device performance, the system comprising:
a camera for recording video data of a patient environment;
at least one processing device; and
at least one computer readable data storage device storing software instructions that, when executed by the at least one processing device, cause the at least one processing device to:
identify a caregiver inside the patient environment based on the video data;
analyze the video data to determine a classification of the caregiver inside the patient environment; and
adjust operation of at least one medical device based on the classification of the caregiver inside the patient environment.
